(19) United States
(12) Patent Application Publication
(10) Pub. No.: US 2014/0257003 A1
(43) Pub. Date: Sep. 11, 2014

(54) CHEMICAL REACTION PROCESS WITH ADDITION OF METAL HALIDES

(71) Applicant: BASF SE, Ludwigshafen (DE)

(72) Inventors: Steffen Tschirschwitz, Mannheim (DE); Stefan Bitterlich, Dirmstein (DE); Nicole Holub, Mannheim (DE); Michael Hübner, Lampertheim (DE)

(73) Assignee: BASF SE, Ludwigshafen (DE)

(21) Appl. No.: 14/199,065

(22) Filed: Mar. 6, 2014

Related U.S. Application Data
(60) Provisional application No. 61/773,841, filed on Mar. 7, 2013.

(51) Int. Cl. C07C 5/29

(52) U.S. Cl. CPC C07C 5/29
USPC 585/372

(57) ABSTRACT

The present invention relates to a chemical reaction process, preferably an isomerization process, of at least one hydrocarbon in the presence of an ionic liquid. The chemical reaction is carried out in an apparatus (V1) with at least one metal halide, preferably aluminum halide, being introduced repeatedly or continuously into the apparatus (V1). The anion of the ionic liquid used comprises at least one metal component and at least one halogen component. Here, the anion of the ionic liquid and the metal halide introduced into the apparatus (V1) have the same halogen component and the same metal component. The ionic liquid used in the respective chemical reaction, in particular in an isomerization, can (inter alia) be regenerated by the process of the invention.

This patent application claims the benefit of pending U.S. provisional patent application Ser. No. 61/773,841 filed on Mar. 7, 2013, incorporated in its entirety herein by reference.

The present invention relates to a chemical reaction process, preferably an isomerization process, of at least one hydrocarbon in the presence of an ionic liquid. The chemical reaction is carried out in an apparatus (V1) with at least one metal halide, preferably aluminum halide, being introduced repeatedly or continuously into the apparatus (V1). The anion of the ionic liquid used comprises at least one metal component and at least one halogen component. Here, the anion of the ionic liquid and the metal halide introduced into the apparatus (V1) have the same halogen component and the same metal component. The ionic liquid used in the respective chemical reaction, in particular in an isomerization, can (inter alia) be regenerated by the process of the invention.

Ionic liquids, in particular acidic ionic liquids, are suitable, inter alia, as catalysts for the isomerization of hydrocarbons. Such a use of an ionic liquid is, for example, disclosed in WO 2011/069929 where a specific selection of ionic liquids is used in the presence of an olefin to isomerize saturated hydrocarbons, in particular to isomerize methylcyclopentane (MCP) to cyclohexane. In the process according to WO 2011/069929 there is no indication that the ionic liquids used in the isomerization are treated in any way with a metal halide. An analogous process is described in WO 2011/069957 but there the isomerization is not carried out in the presence of an olefin but instead using a copper(II) compound.

In general, ionic liquids and hydrocarbons (organic phases) are immiscible or only very sparingly miscible and form two separate phases. To be able to utilize the abovementioned catalytic effect, intensive contact has to be established between organic phase and the ionic liquid. For this purpose, the two phases are frequently mixed in stirred vessels with intensive stirring to give dispersions. Depending on parameters such as type of ionic liquid or of organic phase or the phase ratio, the dispersion can either be present as a dispersion of an ionic liquid in the organic phase or can be a dispersion of the organic phase in the ionic liquid.

Especially in a continuous mode of operation, a partial amount and/or constituents of the ionic liquid used, in particular the anion part, is continually discharged in the form of metal halides such as aluminum chloride and/or hydrogen halides such as HCl via the organic phase in a chemical reaction process, in particular in an isomerization, as a result of which a reduction in the activity of the ionic liquid used, preferably as catalyst, in the chemical reaction process is found.

EP-A 2 455 358 relates to processes for regenerating and maintaining the activity of an ionic liquid used as catalyst, in particular in connection with the preparation of alkylates by means of alkylation reactions. Here, hydrogen halide or halogenated hydrocarbons are added to the catalyst (acidic ionic liquid) in the feed stream during the alkylation reaction. The addition of the hydrogen halide or the halogenated hydrocarbon can also be carried out continuously. Furthermore, EP-A 2 455 358 discloses an analogous process for preparing alkylates by means of an alkylation reaction using isobutene and C4-alkenes as feed stream and acidic ionic liquids as catalyst. However, a metal halide is not added to the acidic ionic liquid used in either of the two processes disclosed in EP-A 2 455 358.

A. Berenblyum (Applied Catalysis. A: General 315 (2006) 128-134) discloses studies on the catalytic activity of chloroaluminate-comprising ionic liquids in connection with the isomerization of heptane. Here, studies are carried out on the solubility of HCl in the chloroaluminate-comprising ionic liquid and on the distribution of aluminum chloride between chloroaluminate-comprising ionic liquid and heptane. In the system examined, HCl is identified as catalytically active component and aluminum chloride is identified as cocatalyst. The decrease in activity of the chloroaluminate-comprising ionic liquid is attributed to the loss of HCl and the formation of an acidic soluble oil which poisons the catalyst. The experiments are (at least partly) carried out with continuous introduction of HCl, but an addition of metal halides such as aluminum chloride is not carried out.

US-A 2010/0065476 discloses methods of measuring and adapting the flow of a halogen-comprising additive in a continuous reactor process, for example in alkylations of olefins or aromatics or in dehydrogenation processes. The halogen-comprising additives can be Brönsted acids such as hydrogen chloride, hydrogen bromide or fluorinated alkanesulfonic acids and also metal halides such as sodium chloride or copper chloride. Furthermore, this document discloses apparatuses for carrying out the corresponding methods, which comprise a reactor comprising an ionic liquid, measurement devices for determining the halogen concentration in the reactor outlet and a control system for controlling the halogen concentration. However, according to US-A 2010/0065476, there is no relationship between the type of metal halide added and the halogen component and metal component comprised in the anion of the ionic liquid.

Rather, the ionic liquids which can be used for the process according to US-A 2010/0065476 are not subject to any restrictions since in principle all known anions such as Cl⁻, NO₃⁻, PF₆⁻ or AlCl₄⁻ can be used in the anion part of the ionic liquids. Therefore, anions which have no halogen component and/or metal component, can also be used. In the two examples, an ionic liquid having Al as metal component and Cl as halogen component is used in an alkylation process, but hydrogen chloride rather than a metal halide is used as halogen-comprising additive. Furthermore, the method described in US-A 2010/0065476 comprises continual sampling and halide analysis of the feed stream to the reaction as necessary constituent. This complicated procedure can in principle be dispensed with in the present invention.

US-A 2007/0249485 discloses a process for regenerating used acidic ionic liquids which have been used as catalyst, where the appropriate ionic liquid is brought into contact with at least one metal in a regeneration zone in the absence of hydrogen. As metal, it is possible to use, for example, aluminum, gallium or zinc, and the ionic liquid is preferably used for catalyzing Friedel-Crafts reactions. An analogous process is disclosed in US-A 2007/0142217, where the regeneration is additionally carried out in the presence of a Brönsted acid such as hydrogen chloride.

WO 2011/006848 discloses a process for modifying an alkylation unit for HF or sulfonic acid and an alkylation unit for ionic liquids. In this process, the ionic liquid used as catalyst is, inter alia, regenerated by adding hydrogen halide or a haloalkane. The use of metal halide is, however, not disclosed.

It is an object of the present invention to provide a novel process for the chemical reaction of at least one hydrocarbon in the presence of an ionic liquid, in particular for isomerization of at least one hydrocarbon in the presence of an ionic liquid.

The object is achieved by a chemical reaction process of at least one hydrocarbon in an apparatus (V1) in the presence of an ionic liquid in which the anion comprises at least one metal component and at least one halogen component, wherein at least one metal halide is introduced repeatedly or continuously into the apparatus (V1) and the anion of the ionic liquid and the metal halide have the same halogen component and the same metal component.

A chemical reaction, in particular an isomerization, of hydrocarbons can be carried out in an advantageous way by means of the process of the invention. Owing to the repeated or continuous addition of at least one metal halide to the ionic liquid present in the apparatus (V1), the catalytic activity of the respective ionic liquid is kept largely constant. The effect can be reinforced further when, in addition to the metal halide, preferably aluminum chloride, a hydrogen halide, in particular hydrogen chloride, is added to the ionic liquid present in the apparatus (V1) or this is continually in contact with a preferably gaseous phase comprising the hydrogen halide.

Furthermore, it is advantageous for the metal halide not to be added directly to the ionic liquid in the apparatus (V1) but for the metal halide rather to be firstly premixed with a main component present in the apparatus (V1) in an apparatus or device (V2) outside the apparatus (V1). This can firstly be the ionic liquid itself which originates from the reaction outlet from the apparatus (V1) and is separated off from the reaction outlet by means of a phase separation unit, preferably a phase separator, and recirculated to the apparatus (V1) (see also FIG. 1 below).

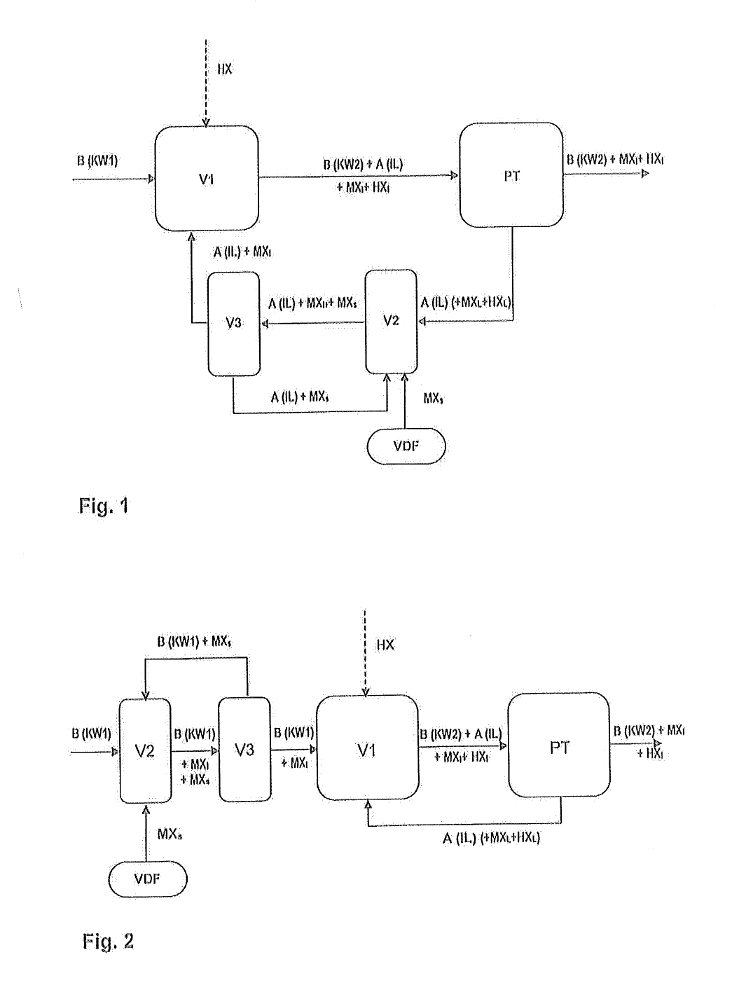

However, it is particularly advantageous to add the metal halide to the feed stream comprising the hydrocarbons which are to be subjected to a chemical reaction, in particular an isomerization, in the apparatus (V1). In this variant, which is also illustrated below in FIG. 2, the apparatus required for addition of the metal halide is simpler because the corresponding apparatus (V2), detached from its specific function, does not have to be made of corrosion-stable material, which is generally necessary in the case of addition to the recirculated ionic liquid or introduction directly into the apparatus (V1) since many ionic liquids are highly corrosive. Furthermore, in the case of addition of the metal halide to the hydrocarbon-comprising stream it is also not necessary for the corresponding apparatus to be designed for high reaction pressures.

The inventive chemical reaction process of at least one hydrocarbon in the presence of an ionic liquid with addition of metal halide is defined in more detail below.

For the purposes of the present invention, a “chemical reaction process” or “chemical reaction” is in principle any chemical reaction known to those skilled in the art in which at least one hydrocarbon is chemically reacted, modified or changed with regard to its composition or structure in another way.

The chemical reaction process is preferably selected from among an alkylation, a polymerization, a dimerization, an oligomerization, an acylation, a metathesis, a polymerization or copolymerization, an isomerization, a carbonylation and combinations thereof. Alkylations, isomerizations, polymerizations, etc., are known to those skilled in the art. For the purposes of the present invention, the chemical reaction process is particularly preferably an isomerization.

For the purposes of the present invention, all ionic liquids known to those skilled in the art in which the anion comprises at least one metal component and at least one halogen component are in principle suitable as ionic liquids. In addition, they can themselves catalyze the reaction carried out in the particular case or have a solvent capability for the catalyst used in the particular case. An overview of suitable ionic liquids may, for the case of isomerization, be found in, for example, WO 2011/069929. For the purposes of the present invention, preference is given to an acidic ionic liquid.

For the purposes of the present invention, the ionic liquid is preferably used as catalyst in a chemical reaction, preferably in an alkylation or isomerization, in particular in an isomerization.

In the (preferably acidic) ionic liquid in the process of the invention, the metal component in the anion of the ionic liquid is preferably selected from among Al, B, Ga, In, Fe, Zn and Ti and/or the halogen component is selected from among F, Cl, Br and I, in particular from among Cl and Br. The (preferably acidic) ionic liquid more preferably has a haloaluminate ion having the composition AlₙX₍₃ₙ₊₁₎ where 1<n<2.5 and X=halogen, preferably X=F, Cl, Br or I, in particular X=Cl, as anion.

All cations known to those skilled in the art are in principle suitable as cations. Examples are an unsubstituted or at least partially alkylated ammonium ion or a heterocyclic (monovalent) cation optionally having alkyl side chains, in particular a pyridinium ion, an imidazolium ion, a pyridazinium ion, a pyrazolium ion, an imidazolinium ion, a thiazolium ion, a triazolium ion, a pyrrolidinium ion, an imidazolidinium ion or a phosphonium ion. The at least partially alkylated ammonium ion preferably comprises one, two or three alkyl radicals (each) having from 1 to 10 carbon atoms. If two or three alkyl substituents are present on the respective ammonium ions, the chain length in each case can be selected independently; preference is given to all alkyl substituents having the same chain length. Particular preference is given to trialkylated ammonium ions having a chain length of from 1 to 3 carbon atoms. The heterocyclic cation is preferably an imidazolium ion or a pyridinium ion.

The ionic liquid preferably has an ammonium ion, more preferably trialkylammonium, as cation and/or a chloroaluminate ion of the composition AlₓCl₃ₓ₊₁ where 1<x<2.5 as anion.

The ionic liquid, in particular the acidic ionic liquid, particularly preferably comprises an at least partially alkylated ammonium ion as cation and a chloroaluminate ion having the composition AlₙCl₍₃ₙ₊₁₎ where 1<n<2.5 as anion. Examples of such particularly preferred ionic liquids are trimethylammonium chloroaluminate and triethylammonium chloroaluminate.

In principle, any hydrocarbons can be comprised in the apparatus (V1) in the process of the invention. A person skilled in the art will know, on the basis of general knowledge in the art, which hydrocarbons in which compositions are best suited for which specific chemical reaction process. Compounds which themselves are not hydrocarbons can optionally also be comprised (in the form of mixtures). In the following text, the composition of the hydrocarbons comprised in the apparatus (V1) will be illustrated by means of the isomerization which is preferred as chemical reaction for the purposes of the present invention.

In the chemical reaction in the apparatus (V1), in particular in the isomerization, preference is given to using methylcyclopentane (MCP) or a mixture of methylcyclopentane (MCP) with at least one further hydrocarbon selected from among cyclohexane, n-hexane, isohexanes, n-heptane, isoheptanes, methylcyclohexane and dimethylcyclopentanes as hydrocarbon. The corresponding hydrocarbons are thus fed into the apparatus (V1).

More preferably, a mixture of methylcyclopentane (MCP) with at least one further hydrocarbon selected from among cyclohexane, n-hexane, isohexanes, n-heptane, isoheptanes, methylcyclohexane and dimethylcyclopentanes, where the concentration ratio of MCP/cyclohexane is preferably at least 0.2, is used in the chemical reaction, in particular in the isomerization.

For the purposes of the present invention, particular preference is given to isomerizing methylcyclopentane (MCP) to cyclohexane.

The chemical reaction, in particular the isomerization, in the process of the invention preferably gives cyclohexane or a mixture of cyclohexane with at least one further hydrocarbon selected from among methylcyclopentane (MCP), n-hexane, isohexane, n-heptane, isoheptane, methylcyclohexane and dimethylcyclopentane as hydrocarbon.

The chemical reaction, in particular the isomerization, particularly preferably gives a mixture of cyclohexane, MCP and at least one further hydrocarbon. The further hydrocarbon is preferably selected from among n-hexane, isohexane, n-heptane, isoheptane, methylcyclohexane and dimethylcyclopentane. Furthermore, preference is given to a smaller proportion of MCP and open-chain linear hydrocarbons being present in the mixture preferably comprised in the phase (B) described further below and obtained after the isomerization in the process of the invention compared to the corresponding composition of the hydrocarbons or of the phase (B) before the isomerization.

For the purposes of the present invention, the chemical reaction, preferably the isomerization, is carried out in an apparatus (V1) known to those skilled in the art. Suitable apparatuses (V1) are, for example, reactors, other reaction apparatuses, stirred vessels or a cascade of stirred vessels. The apparatus (V1) is preferably a reactor or a cascade of stirred vessels.

Furthermore, at least one metal halide is introduced repeatedly or continuously into the apparatus (V1) in the process of the invention. As indicated above, the anion of the ionic liquid and the metal halide have the same halogen component and the same metal component. In principle, all metal halides which are known to those skilled in the art and satisfy this criterion are suitable. The metal halide is preferably selected from among AlX₃, BX₃, GaX₃, InX₃, FeX₃, ZnX₂ and TiX₄ where X=halogen, preferably X=Cl or Br, even more preferably X=Cl. The metal halide is in particular AlCl₃.

If, for example, the ionic liquid used in the apparatus (V1) comprises Al₂Cl₇⁻ as anion, AlCl₃ can correspondingly be used as metal halide. In the case of mixed-component anions such as Al₂BrCl₆⁻, it is possible to use, for example, a corresponding mixture of AlCl₃ and AlBr₃. This also applies analogously in respect of the choice of the appropriate metal component of the metal halide used when the metal component of the anion of the respective ionic liquid comprises two or more components such as Al or Cu.

The addition of at least one metal halide to the apparatus (V1) can be carried out repeatedly or continuously, as mentioned above. Here, the metal halide can be added in liquid or solid form. It has also been found that the metal halide does not have to be introduced directly into the apparatus (V1) but the metal halide can instead firstly be added to one or more of the components participating in the chemical reaction process in another apparatus, for example in a contact apparatus (V2). From this other apparatus, the metal halide is conveyed into the apparatus (V1) (indirect addition of the metal halide to (V1)). The transfer or conveying of the metal halide from the other apparatus into the apparatus (V1) is effected by the methods known to those skilled in the art, for example using pumps.

The two embodiments defined in more detail in the following text in combination with the FIGS. 1 and 2 are preferred for the addition of the metal halide. Both embodiments are an indirect addition in which the metal halide is firstly introduced into the system via the contact apparatus (V2) from where it goes into the apparatus (V1).

For the purposes of the present invention, “continuous addition” of the metal halide means that the corresponding addition occurs over a relatively long period of time, preferably over at least 50%, more preferably over at least 70%, even more preferably over at least 90%, of the reaction time, in particular over the entire reaction time. The continuous addition is preferably carried out with the corresponding apparatus for introduction (addition) of the metal halide (e.g. a star feeder) being in operation over the abovementioned periods of time.

For the purposes of the present invention, “repeated addition” of the metal halide means that the corresponding addition is carried out at regular or irregular time intervals. The corresponding addition is preferably triggered by the occurrence of an addition condition described below, in particular in connection with the saturation concentration in the phase (B). The time intervals between the individual additions are at least one hour, preferably at least one day. For the purposes of the present invention, the term “repeated” again means at least two, for example 3, 4, 5, 10 or even 100, individual additions. The actual number of the individual additions depends on the operating time. This ideally tends toward infinity.

In other words, repeated addition of the metal halide means, for the purposes of the present invention, the addition at separate times of a number of batches of metal halide. The addition of an individual batch can take from a number of seconds to a number of minutes, and somewhat longer periods are optionally also considerable. According to the invention, the time interval between the respective addition of an individual batch is at least ten times as great as the duration of the addition of the corresponding batch. For the purposes of the present invention, the embodiment of “repeated addition” can optionally be combined with the embodiment of “continuous addition”.

For the purposes of the present invention, the addition of the metal halide is particularly preferably carried out in such a way that a concentration of ≧70%, preferably ≧90%, of the saturation concentration of the metal halide is established in the apparatus (V1). It is also possible for supersaturation of metal halide to occur in the apparatus (V1). If this is the case, an (additional) solid phase of metal halide is formed in the apparatus (V1). A concentration of ≧70%, preferably ≧90%, of the saturation concentration of the metal halide is preferably set in the phase (B) (described below). Here, the term “saturation concentration” is as defined in IUPAC: Compendium of Chemical Terminology, 2^{nd} edition (the “Gold Book”), compiled by A. D. McNaught and A. Wilkinson. Blackwell Scientific Publications, Oxford (1997).

In the case of a repeated addition of the metal halide, the next addition in each case is preferably carried out in such a way that a concentration of ≧70%, preferably ≧90%, of the saturation concentration of the metal halide is established again in the apparatus (V1), preferably in the phase (B). The next addition in each case of metal halide is thus carried out when the metal halide concentration has gone below the above limit values.

In particular, the repeated addition of the metal halide is carried out in such a way that the abovementioned saturation-based concentrations of metal halide in the phase (B) are maintained continuously. The next addition in each case of metal halide is thus carried out before the metal halide concentration has gone below the above limit values.

The continuous addition of the metal halide is preferably carried out in such a way that a concentration of ≧70%, preferably ≧90%, of the saturation concentration of the metal halide is maintained continuously in the apparatus (V1). In particular, this is maintained in the phase (B) (described further below).

In a preferred embodiment of the present invention, two phases (A and B) are comprised in the apparatus (V1); further phases can optionally also be comprised. The phase (A) comprises at least one ionic liquid as per the description given above, with the proportion of ionic liquid in the phase (A) being greater than 50% by weight. The phase (A) is preferably a phase which comprises ionic liquids and is immiscible or only sparingly miscible with hydrocarbons and/or comprises not more than 10% by weight of hydrocarbons.

For example, mixtures of two or more ionic liquids can be comprised in the phase (A); the phase (A) preferably comprises one ionic liquid. Apart from the ionic liquid, further components which are miscible with the ionic liquid can also be comprised in the phase (A). These can be hydrocarbons from the phase (B) described below, which generally have a limited solubility in ionic liquids. Furthermore, phase (A) can also comprise cocatalysts which are employed in isomerization reactions using ionic liquids. A preferred example of such cocatalysts is hydrogen halides, in particular hydrogen chloride. In addition, constituents or decomposition products of the ionic liquids, which can be formed, for example, during the isomerization process, can also be comprised in the phase (A). The proportion of ionic liquid in phase (A) is preferably greater than 80% by weight.

For the purposes of the present invention, the phase (B) comprises at least one hydrocarbon, with the content of hydrocarbon in the phase (B) being greater than 50% by weight. The phase (B) is preferably a hydrocarbon-comprising phase which is immiscible or only sparingly miscible with ionic liquids and/or comprises not more than 1% by weight of ionic liquids (based on the total weight of the phase).

The specific composition of the phase (B) is dependent on the chemical reaction process selected. The phase (B) experiences a change in its composition during the course of a chemical reaction process. The specific hydrocarbons which can be comprised in the phase (B) before and after the chemical reaction, in particular the isomerization, are described below.

Furthermore, preference is given to the ionic liquid in the apparatus (V1) being comprised in a proportion of greater than 50% by weight in a phase (A) which has a higher viscosity than a phase (B) in which at least one hydrocarbon is comprised in a proportion of greater than 50% by weight and the phases (A) and (B) being in direct contact with one another, for example by forming a heterogeneous mixture with one another.

In an embodiment of the present invention, the chemical reaction, in particular the isomerization, occurs in a dispersion (D1) in which the phase (B) is dispersed in the phase (A). The dispersion direction (i.e. the information as to which phase is present in disperse form in the other phase) can be determined by examining a sample, optionally after addition of a dye which selectively colors one phase, in transmitted light under an optical microscope. The phases (A) and (B) have the above definitions.

The dispersion (D1) can be produced by methods known to those skilled in the art; for example, such a dispersion can be generated by intensive stirring of the phases. In a further embodiment of the present invention, the volume ratio of the phase (A) to phase (B) in the dispersion (D1) is in the range from 2.5:1 to 4:1 [vol/vol], preferably in the range from 2.5:1 to 3:1 [vol/vol].

In an embodiment of the present invention, at least one hydrogen halide (HX), preferably hydrogen chloride (HCl), is introduced into the apparatus (V1), with the introduction of hydrogen halide preferably being carried out repeatedly or continuously. The repeated or continuous introduction of the hydrogen halide is carried out in a manner analogous to the above-described repeated or continuous addition of the metal halide.

The hydrogen halide (HX) is preferably introduced in gaseous form into the apparatus (V1), preferably by setting a constant HX partial pressure over the ionic liquid, preferably at a constant HX partial pressure of from 0.5 to 10 bara, more preferably from 1 to 5 bara.

If gaseous hydrogen halide (HX) is introduced into the apparatus (V1), a mixture comprising the following phases:
i) the phase (A) comprising the ionic liquid,
ii) the phase (B) comprising at least one hydrocarbon,
iii) optionally the phase (C) comprising solid metal halide, preferably solid AlX₃, and
iv) the phase (D) comprising gaseous HX,
   can be obtained in an embodiment of the present invention.

The process of the invention, in particular the isomerization, is preferably carried out continuously. The compounds (products) formed in the chemical reaction, in particular in the isomerization, can be discharged from the apparatus (V1) by methods known to those skilled in the art.

For example, a stream comprising the phase (B) and the phase (A), with at least one hydrocarbon which was prepared in the chemical reaction being comprised in the phase (B), can be discharged from the apparatus (V1) in which the chemical reaction is carried out. This stream is in turn preferably introduced into a phase separation apparatus (phase separation unit). Phase separation apparatuses per se are known to those skilled in the art. This phase separation apparatus is preferably a phase separator.

The apparatus (V1) is preferably a reactor or a cascade of stirred vessels, and a phase separation apparatus, preferably a phase separator, is located downstream of the apparatus (V1).

Furthermore, the phase (A) comprising the ionic liquid is preferably separated off from the phase (B) comprising at least one hydrocarbon in the phase separation apparatus, with the phase (A) preferably being recirculated to the apparatus (V1), in particular to the reactor or to the starting point of the cascade of stirred vessels.

In the phase separation apparatus, preference is given to a first stream comprising at least 70% by weight, preferably at least 90%, of the phase (A) and a second stream comprising at least 70%, preferably at least 90%, of the phase (B) being separated from one another. The above figures in % are based on the corresponding amounts comprised in the stream introduced into the phase separation apparatus.

Furthermore, preference is given, for the purposes of the present invention, to a contact apparatus (V2) which is preferably a moving bed, a fluidized bed or a stirred vessel being installed upstream of the apparatus (V1), with the metal halide firstly being introduced into the contact apparatus (V2) and from there being conveyed into the apparatus (V1). The metal halide can be added in solid or liquid form, particularly preferably in solid form.

An apparatus (V3) for solid/liquid or liquid/liquid separation, which is preferably a phase separator, a gravity separator, a hydrocyclone, an apparatus having a dead-end filter or a crossflow filter, can in turn be installed downstream of the contact apparatus (V2). The apparatus (V3) for solid/liquid or liquid/liquid separation is optionally integrated into the contact apparatus (V2) as part of the latter apparatus, for example by (V2) being a stirred vessel which comprises a stirring zone and, arranged above this, a disengagement zone in which gravity-induced separation of solid and liquid takes place. A stream which has been separated off in the apparatus (V3) for solid/liquid or liquid/liquid separation and is enriched in solid is preferably recirculated to the contact apparatus (V2).

Regardless of the presence of a downstream apparatus (V3) for solid/liquid or liquid/liquid separation, preference is given in relation to the contact apparatus (V2) to the metal halide being introduced into the contact apparatus (V2) repeatedly or continuously by means of an apparatus for the metering or transport of solid or liquid; in the case of solid, preferably by means of a star feeder or pneumatic transport; in the case of liquid, preferably by means of a pump.

Preference is likewise given to a liquid which comprises the materials to be reacted in the apparatus (V1) and/or which is fed into the apparatus (V1) being passed through the contact apparatus (V2). These two variants will be explained in more detail below in connection with FIGS. 1 and 2.

In a preferred embodiment, the presence of a second, in particular solid, phase in the contact apparatus (V2) is continually monitored visually or by means of another suitable apparatus or process, preferably by means of a turbidity measurement, and when the second phase disappears metal halide is introduced into the contact apparatus (V2) by means of an apparatus for the metering or transport of solid.

In a preferred embodiment of the present invention, the recirculated phase (A) which originates from the above-described phase separation apparatus, in particular the phase separator, is passed through the contact apparatus (V2) and (V2) is located between phase separation apparatus and apparatus (V1), with an apparatus (V3) for solid/liquid separation or liquid/liquid separation optionally being installed downstream of (V2).

In FIG. 1, the process of the invention according to a preferred embodiment, which is preferably carried out as an isomerization, is illustrated again. “MX” denotes metal halide, “s” means solid and “I” means liquid or dissolved. “IL” denotes ionic liquid, “VDF” denotes apparatus for metering or transporting solid. “A” denotes phase (A), with the respective main component of this phase being placed in parentheses (in the present case ionic liquid). “B” denotes phase (B), with “KW1” denoting a first hydrocarbon mixture and “KW2” denoting a second hydrocarbon mixture which is formed from KW1 in a chemical reaction, preferably an isomerization, in the apparatus (V1).

The phase separation unit (PT) is preferably a phase separator, and the apparatus (V1) is preferably a reactor or a stirred vessel or a cascade of stirred vessels. FIG. 1 also shows an apparatus (V3) for solid/liquid separation or liquid/liquid separation, from which a stream enriched in solid (i.e. MX) is recirculated to the contact apparatus (V2). The embodiment shown in FIG. 1 can optionally also be carried out without the apparatus (V3) and the corresponding return line to the apparatus (V2). Complete removal of solid (MX_{S}) is preferably carried out in the apparatus (V3). The phase (A) recirculated from the phase separation unit (PT) preferably comprises, apart from the ionic liquid, also HX and MX (in dissolved form) in a lower concentration compared to the stream which is introduced into the phase separation unit (PT). Furthermore, hydrogen halide, preferably hydrogen chloride, can optionally also be introduced into the apparatus (V1) in this embodiment. Aluminum chloride is preferably used as metal halide in this embodiment.

In a further preferred embodiment of the present invention, a liquid comprising the phase (B), particularly preferably the feed mixture for the reaction to be carried out, is passed through the contact apparatus (V2) and (V2) is installed upstream of the apparatus (V1), with an apparatus (V3) for solid/liquid separation or liquid/liquid separation optionally being installed downstream of (V2).

The above-described further embodiment of the present invention will be additionally illustrated below in a preferred embodiment in connection with FIG. 2. In FIG. 2, the abbreviations, arrows and other symbols have a meaning analogous to that indicated above for FIG. 1 or in the description of this preferred embodiment. In the embodiment according to FIG. 2, the metal halide, preferably aluminum chloride, is then introduced into the hydrocarbon-comprising stream (phase (B)) fed into the apparatus (V1). As indicated above in connection with FIG. 1, the use of the apparatus (V3) and the associated return line is not absolutely necessary in the embodiment according to FIG. 2, either.

For the purposes of the present invention, cyclohexane is preferably isolated from the output from the apparatus (V1), in particular from the hydrocarbon-comprising output from a phase separation unit, preferably a phase separator, installed downstream of the apparatus (V1). Processes and apparatuses for separating cyclohexane from such an output or stream, in particular when the stream concerned is a hydrocarbon mixture, are known to those skilled in the art. Further purification steps (for example scrubbing with an aqueous and/or alkaline phase) which are known to those skilled in the art can optionally be carried out before the isolation of cyclohexane.

The present invention is illustrated below by the examples.

### GENERAL EXPERIMENTAL PROCEDURE

Substances and compositions used for the experiments are as follows:

Ionic liquid (A) with the composition (CH₃)₃NHAl₂Cl₇; a hydrocarbon mixture (B) with the components methylcyclopentane, cyclohexane, n-hexane and isohexanes; gaseous HCl; and solid AlCl₃.

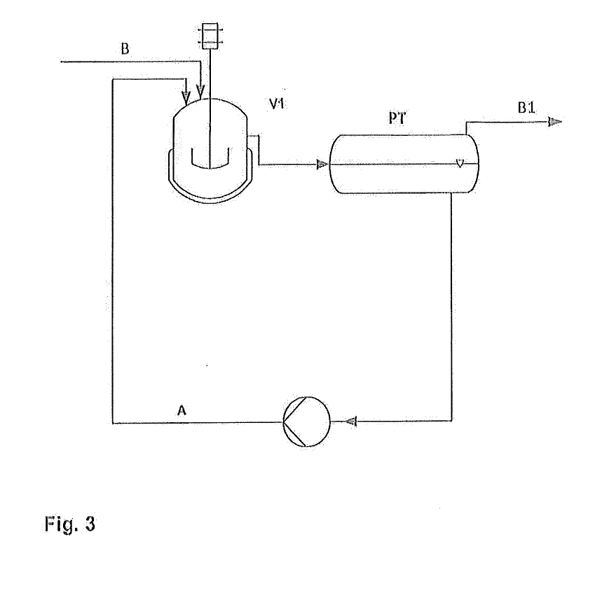

The experimental setup is shown in FIG. 3.

The hydrocarbon mixture B is introduced into a stirred vessel (V1) in which there is a defined amount of ionic liquid. Depending on the experiment, this liquid can be saturated with solid AlCl₃. The reaction of the hydrocarbon mixture—an isomerization of methylcyclopentane to cyclohexane—takes place in this vessel. The isomerized hydrocarbon mixture is referred to as B1. The fill level of (V1) is regulated here by adjustment of the variable overflow between V1 and PT. The dispersion of A in B is passed into the phase separator (PT), in which the two phases separate. The ionic liquid, as the heavier phase (A), is obtained as the bottom phase, and is conveyed by a pump back into the container V1. The top, organic phase is drawn off and analyzed for its composition by gas chromatography. Additionally, with gaseous HCl, an overpressure of 2 bar is set in the system.

### Example 1

### General

Organic/IL ratio=1/5 to 1/4 vol/vol

Temperature=50° C.

p=3 bar abs

Reactor volumes=about 160 mL

IL amount: 180 g

Amount of added AlCl₃: 10 g

HCl supply: 1 L/h (stp)

Space velocity=0.5-0.7 m³_{org}/m³_{IL} hr

c_{MCP,in}=about 20% by weight

### Procedure

The reactor is charged with the IL (ionic liquid), and 10 g of AlCl₃ are suspended therein. A hydrocarbon mixture containing about 20% by weight of MCP (cyclohexane 50%, n-hexane 28% and isohexane 2%, in each case by weight) is run continuously into the reactor (V1) and separated off again in a phase separator. With time, AlCl₃ is discharged from the reactor by dissolution in the hydrocarbon mixture. If there is no longer any AlCl₃ in suspension in the IL, further 10 g portions of AlCl₃ are added (about every 500 hours).

### Results

| Experiment duration [h] | MCP conversion [%] |
|---|---|
| 24 | 34.7 |
| 100 | 34.5 |
| 300 | 35.4 |
| 1000 | 35.2 |
| 1500 | 35.6 |
| 2500 | 36.1 |
| 3000 | 35.1 |

Through the continual addition of AlCl₃ it is possible to achieve a constant MCP conversion over a long time period. On the basis of the lower initial concentration of MCP in the feed, therefore, the conversion is lower in comparison to the initial conversion in the comparative example.

### Example 2

### Comparative

### General

Organic/IL ratio=1/5 to 1/4 vol/vol

Temperature=60° C.

p=3 bar abs

Reactor volumes=about 250 mL

IL amount: 260 g

Amount of added AlCl₃: 0 g

HCl supply: 0 L/h (stp)

Space velocity=0.2 m³_{org}/m³_{IL} hr

c_{MCP,in}=about 45% by weight

### Procedure

The reactor is charged with the IL. A hydrocarbon mixture containing about 51% by weight of MCP (cyclohexane 19%, n-hexane 29% and isohexane 1%, in each case by weight) is run continuously into the reactor (V1) and separated off again in a phase separator.

### Results

| Experiment duration [h] | MCP conversion [%] |
|---|---|
| 24 | 47.2 |
| 100 | 35.7 |
| 200 | 30.1 |
| 300 | 25.8 |
| 400 | 26.1 |
| 500 | 25.0 |
| 600 | 20.8 |
| 700 | 18.1 |

Following initial high activity, the MCP conversion drops over the course of 700 hours to less than 20%.

1-19. (canceled)

20. A chemical reaction process of at least one hydrocarbon in an apparatus (V1) in the presence of an ionic liquid in which the anion comprises at least one metal component and at least one halogen component, wherein at least one metal halide is introduced repeatedly or continuously into the apparatus (V1) and the anion of the ionic liquid and the metal halide have the same halogen component and the same metal component.

21. The process according to claim 20, wherein
i) the metal component in the anion of the ionic liquid is selected from among Al, B, Ga, In, Fe, Zn and Ti or the halogen component is selected from among F, Cl, Br and I, or
ii) the metal halide is selected from the group consisting of AlX₃, BX₃, GaX₃, InX₃, FeX₃, ZnX₂, TiX₄, and combinations thereof, where X=halogen.

22. The process according to claim 20, wherein the ionic liquid in the apparatus (V1) comprises greater than 50% by weight of a phase (A) which has a higher viscosity than a phase (B) in which at least one hydrocarbon is comprised in a proportion of greater than 50% by weight and the phases (A) and (B) are in direct contact with one another.

23. The process according to claim 20, wherein a concentration of ≧70% of the saturation concentration of the metal halide is set in the apparatus (V1).

24. The process according to claim 23, wherein the concentration is ≧90% or the concentration is set in the phase (B).

25. The process according to claim 20, wherein, in the case of a repeated addition of the metal halide, the next addition in each case is carried out in such a way that a concentration of ≧70% of the saturation concentration of the metal halide is established again in the apparatus (V1).

26. The process according to claim 25, wherein the concentration is maintained continuously.

27. The process according to claim 20, wherein the continuous addition of the metal halide is carried out in such a way that a concentration of ≧70% of the saturation concentration of the metal halide is maintained continuously in the apparatus (V1).

28. The process according to claim 20, wherein at least one hydrogen halide (HX) is introduced into the apparatus (V1).

29. The process according to claim 28, wherein HX is hydrogen chloride (HCl) or the introduction of hydrogen halide being carried out repeatedly or continuously.

30. The process according to claim 20, wherein the ionic liquid has a haloaluminate ion having the composition AlₙX₍₃ₙ₊₁₎ where 1<n<2.5 and X=halogen as anion.

31. The process according to claim 30, wherein the ionic liquid has an ammonium ion as cation or a chloroaluminate ion of the composition AlₙCl₍₃ₙ₊₁₎, where 1<n<2.5 as anion.

32. The process according to claim 20, wherein the ionic liquid is used as catalyst in a chemical reaction.

33. The process according to claim 32, wherein the chemical reaction is an isomerization.

34. The process according to claim 20, wherein a contact apparatus (V2) is installed upstream of the apparatus (V1), with the metal halide firstly being introduced into the contact apparatus (V2) and from there conveyed into the apparatus (V1).

35. The process according to claim 34, wherein the contact apparatus (V2) is a moving bed, a fluidized bed or a stirred vessel.

36. The process according to claim 34, wherein an apparatus (V3) for solid/liquid separation or liquid/liquid separation is installed downstream of the contact apparatus (V2), where the apparatus (V3) for solid/liquid separation or liquid/liquid separation is optionally integrated into the contact apparatus (V2) as part of the latter apparatus and a stream which has been separated off in the apparatus (V3) for solid/liquid separation or liquid/liquid separation and is enriched in solid is recirculated to the contact apparatus (V2).

37. The process according to claim 36, wherein the apparatus (V3) is a phase separator, a gravity separator, a hydrocyclone, an apparatus having a dead-end filter or a crossflow filter.

38. The process according to claim 34, wherein the metal halide is introduced into the contact apparatus (V2) repeatedly or continuously by means of an apparatus for the metering or transport of solid.

39. The process according to claim 34, wherein the presence of a second phase in the contact apparatus (V2) is continually monitored visually or by means of another suitable apparatus or process, preferably by means of a turbidity measurement, and when the second phase disappears metal halide is introduced into the contact apparatus (V2).

40. The process according to claim 39, wherein the second phase is a solid phase.

41. The process according to claim 34, wherein the metal halide is introduced as a suspension into the contact apparatus (V2).

42. The process according to claim 34, wherein a liquid which comprises the materials to be reacted in the apparatus (V1) or which is fed into the apparatus (V1) is passed through the contact apparatus (V2).

43. The process according to claim 20, wherein the apparatus (V1) is a reactor or a cascade of stirred vessels, or a phase separation apparatus is located downstream of the apparatus (V1).

44. The process according to claim 43, wherein the phase (A) comprising the ionic liquid is separated off from the phase (B) comprising at least one hydrocarbon in the phase separation apparatus, with the phase (A) being recirculated to the apparatus (V1).

45. The process according to claim 44, wherein the phase (A) is recirculated to the reactor or to the starting point of the cascade of stirred vessels.

46. The process according to claim 44, wherein the recirculated phase (A) is passed through the contact apparatus (V2) and (V2) is located between phase separation apparatus and apparatus (V1), with an apparatus (V3) for solid/liquid separation or liquid/liquid separation optionally being installed downstream of (V2).

47. The process according to claim 46, wherein a liquid comprising the phase (B) is passed through the contact apparatus (V2) and (V2) is installed upstream of the apparatus (V1), with an apparatus (V3) for solid/liquid separation or liquid/liquid separation optionally being installed downstream of (V2).
